(19) United States
(12) Patent Application Publication
(10) Pub. No.: US 2006/0018827 A1
(43) Pub. Date: Jan. 26, 2006

(54) Positron therapy of inflammation, infection and disease

(76) Inventors: Ekaterina Dadachova, Mahopac, NY (US); Renee M. Moadel, Great Neck, NY (US)

Correspondence Address: AMSTER, ROTHSTEIN & EBENSTEIN LLP, 90 PARK AVENUE, NEW YORK, NY, 10016, US

(21) Appl. No.: 11/177,246

(22) Filed: Jul. 7, 2005

Related U.S. Application Data
(60) Provisional application No. 60/586,366, filed on Jul. 8, 2004.

(51) Int. Cl. A61K 51/00; A61K 31/70

(52) U.S. Cl. 424/1.11; 514/23

(57) ABSTRACT

This invention provides methods for positron therapy of inflammation, infection and disease which comprise administering to a subject a positron emitter or an agent labeled with a positron emitter, wherein the positron emitter or agent binds to or is taken up by affected tissue or by cells at the site of affected tissue, infection or inflammation. The invention also provides kits, compositions and methods of making compositions comprising positron emitters for the treatment of inflammation, infection and disease.

### CROSS-REFERENCE TO RELATED APPLICATION

This application claims the benefit of U.S. Provisional Patent Application No. 60/586,366, filed Jul. 8, 2004, the content of which is hereby incorporated by reference.

### STATEMENT OF GOVERNMENT SUPPORT

The invention disclosed herein was made with U.S. Government support under grant number P30CA013330-319017 from the National Cancer Institute, U.S. Department of Health and Human Services. Accordingly, the U.S. Government has certain rights in this invention.

### FIELD OF THE INVENTION

The present invention relates to the use of positrons for the treatment of inflammation, infection and/or disease, where a positron emitter, or an agent bound to a positron emitter, binds to and/or is taken up by tissue affected by the inflammation, infection and/or disease, and/or binds to and/or is taken up by cells, such as reticuloendothelial cells, white blood cells and/or phagocytic cells, at the site of the affected tissue, infection and/or inflamation.

### BACKGROUND OF THE INVENTION

Throughout this application various publications are referred to in parenthesis. Full citations for these references may be found at the end of the specification immediately preceding the claims. The disclosures of these publications are hereby incorporated by reference in their entireties into the subject application to more fully describe the art to which the subject application pertains.

Major health problems continue to arise due to infection and disease, such as autoimmune, congenital, genetic, idiopathic, vascular, endocrine, metabolic, hematologic, infectious, inflammatory, nutritional, toxin and/or drug induced, psychiatric, neurologic, immune and malignant disease. Cancer, for example, is still a major cause of death in the developed world. Novel therapeutic modalities are needed for patients in whom standard therapies such as chemotherapy, hormonal treatment and external radiation therapy are not effective.

Due to the early success of radiotherapy of thyroid cancer with ¹³¹I which emits electrons (Seidlin et al., 1946), further development of radiopharmaceuticals for cancer therapy concentrated on electron-emitting radioisotopes. None of the effective radionucleotide therapies that have been developed (e.g., 131-I, 125-I, 89-Sr, 90-Y, 191-Ir) has a positron component.

¹⁸F-2-deoxy-2-fluoro-D-glucose (¹⁸F-FDG) is widely used in positron emission tomography (PET) imaging for the evaluation of patients with tumors (Di Chiro et al. 1982, Hustinx et al. 2002). ¹⁸F-FDG is an analogue of glucose in which the hydroxy group in the 2-position has been replaced with a fluorine atom. In recent years ¹⁸F-FDG-PET has become a well-established modality in diagnosis of breast cancer as many breast cancers demonstrate high avidity for ¹⁸F-FDG (Bombardieri and Crippa, 2001). However, there have not been any indications in the literature of a therapeutic potential of imaging doses of ¹⁸F-FDG.

It has been reported in abstract form that high dose injection of ¹⁸F-FDG directly into a glioma cell xenograft in mice has an effect on the glioma (Meyer et al. 1996, 1998). However, since it is well known that 2-deoxy-D-glucose itself has a tumoricidal effect (Laszlo et al., 1960), the effectiveness of positron therapy in these preliminary reports was not established.

The transport of glucose into cells is mediated by a family of homologous glucose transport proteins which differ in their tissue distribution and physiological properties (Pessin and Bell, 1992). Of these isoforms, GLUT1, an insulin independent transporter, in particular has been found to be expressed at high levels in a variety of cancers, including breast cancer (Aloj et al., 1999; Brown and Wahl, 1993; Younes et al., 1995, 1997; Zamora-Leon et al., 1996). The GLUT profiles of many breast/mammary cancers have been described (Brown et al., 2002; Rivenzon-Segal et al, 2000; Rogers et al., 2003; Zamora-Leon et al., 1996).

### SUMMARY OF THE INVENTION

The present invention is directed to the use of positron emitters for the treatment of disease, infection and inflammation. This treatment is herewith termed “POSITHERAPY” or “POSI-THERAPY”. The invention provides methods of treatment which comprise administering to a subject an amount of positron emitter effective to treat the subject's disease, inflammation and/or infection. The positron emitter can be bound to a delivery agent and/or can administered in the absence of being bound to a delivery agent. The positron emitter and/or an agent labeled with the positron emitter, binds to and/or is taken up by the inflamed, infected and/or diseased tissue, and/or binds to and/or is taken up by cells at the site of inflamed, infected and/or diseased tissue, thereby treating the inflammation, infection and/or disease. The invention also provides kits, compositions and methods of making compositions comprising positron emitters for the treatment of disease.

### BRIEF DESCRIPTION OF THE FIGURES

FIGS. 1A-1D. Coronal FDG-PET images of mice acquired 1 hour after i.p. injection of ¹⁸F-FDG. Slices (2 mm thick) with the highest activity in the tumor were chosen. All mice were lying on their backs with the heads pointing up. Tumors are marked with arrows. Because ¹⁸F-FDG is normally excreted in the urine, bladder activity is visible in all mice. A: PyMT mouse with an abdominal mammary tumor. B: PyMT mouse with a thoracic mammary tumor. C: MMTV-neuT mouse with a thoracic mammary tumor. D: Tumor-free MMTV neuT mouse: no abnormal uptake is seen.

FIG. 2A-2G. Apoptosis and necrosis caused by treatment with ¹⁸F-FDG in mammary tumors in PyMT mice. Apoptotic and necrotic cells were detected with an Apoptag Plus apoptosis detection kit. A: A treated tumor in a 10-week-old mouse has numerous apoptotic cells present in the center of the tumor. B: An untreated tumor in a mouse of the same age has an insignificant number of apoptotic cells. C: Healthy mammary glands of C3H/B6 mice treated with 3 mCi of ¹⁸F-FDG: no apoptotic or necrotic cells are present. D: A treated tumor in a 22-week-old mouse shows extensive necrotic areas; some apoptotic cells are also seen at the rim of the necrotic area. E: An untreated tumor in a 22-week-old mouse. F: An H&E-stained treated tumor in a 22-week-old mouse: heavy infiltration by leukocytes and a loss of coherence of the tumor cells with breaks in the epithelial sheets are observed. G: An untreated tumor in a mouse of the same age shows solid sheets of tumor cells. Original magnifications: (A-C)×400; (D-G)×200.

FIG. 3A-3B. Coronal FDG-PET (A) and CT (B) images of a nude mouse with xenografted Notch tumor in its flank acquired 1 hour after i.p. injection of ¹⁸F-FDG. The slice (2 mm thickness) with the highest activity in the tumor was chosen. The mouse is lying on its back with its head pointing up. Tumor is marked with an arrow on the PET and CT images. As ¹⁸F-FDG is normally excreted in the urine, bladder activity is visible. On the CT, the tumor is visualized as soft tissue fullness as compared with the normal contra-lateral flank.

FIG. 4. Kaplan-Meier survival curves of Notch-tumor bearing mice treated with 2.5 mCi ¹⁸F-FDG in comparison with non-treated mice.

FIG. 5. H&E stained section of Notch tumor from a nude mouse showing vascularity.

FIG. 6A-6C. Expression of GLUT transporters in Notch cells and tumors: A) immunoblot determination of GLUT1, 2, 4 and 8 expression in Notch cells (left lane) and Notch tumors (right lane); B) immunofluorescence of GLUT1 and GLUT8 in Notch tumor; C) H&E-stained consecutive section. Viable malignant cells surrounding blood vessels are marked with a circle, the area of malignant cells with central necrosis is marked with an arrow.

FIG. 7. ¹⁸F-FDG-PET projection image of a patient with breast cancer metastatic to the right lung. A metastasis to the left lung is also visible. Normal activity is observed in the heart, liver, kidneys, bladder, and bowel. The primary breast cancer had previously been resected, and the residual normal non-lactating breast tissue did not display increased ¹⁸F-FDG uptake over the background. The maximum Standardized uptake value (SUV) (right lung lesion) is 9.9.

FIG. 8. FDG-PET scan of patient with metastatic non-small cell lung cancer where the maximum SUV (right mediastinum) is 14.8.

FIG. 9. FDG-PET scan of patient with metastatic esophageal cancer where the maximum SUV (abdominal metastasis) is 33.

### DETAILED DESCRIPTION OF THE INVENTION

The subject invention is directed to a method for treating an inflammation, an infection and/or a disease in a subject, which comprises administering to the subject an amount of a positron emitter effective to treat the inflammation, infection, and/or disease, wherein the positron emitter and/or an agent labeled with the positron emitter, binds to and/or is taken up by inflamed, infected and/or diseased tissue, and/or binds to and/or is taken up by cells at a site of inflamed, infected and/or diseased tissue, thereby treating the inflammation, infection and/or disease.

The positron emitter can be administered to the subject in a form where the positron emitter is not bound to a delivery agent and/or in a form where an agent is bound to the positron emitter. The agent can be used to target the delivery of the positron emitter to particular tissues, such as tumors, or to sites of disease, infection and/or inflammation. The positron emitter, and/or the agent labeled with the positron emitter, can bind to and/or be taken up by inflamed, infected and/or diseased tissue. The positron emitter, and/or the agent labeled with the positron emitter, can also bind to and/or be taken up by cells at the site of inflamed, infected and/or diseased tissue. Such cells include, but are not limited to, reticuloendothelial cells, macrophages, kupfer cells, white blood cells, granulocytes, monocytes, B-lymphocytes, T-lymphocytes, fibroblasts, vascular endothelium cells, phagocytic cells, and/or mononuclear phagocytic cells.

The subject's disease can be an autoimmune, genetic, idiopathic, vascular, endocrine, metabolic, hematologic, infectious, inflammatory, nutritional, toxin- and/or drug- induced, psychiatric, neurologic, immune and/or malignant disease. Autoimmune diseases include, but are not limited to, systemic lupus erythematosis. Genetic diseases include, but are not limited to, sickle cell disease and cystic fibrosis. Idiopathic diseases include, but are not limited to, pneumonitis, renal tubulointerstial disease and amyloidosis. Vascular diseases include, but are not limited to, atherosclerosis, coronary artery disease and peripheral vascular disease. Endocrine diseases include, but are not limited to, hyperthyroidism and pheochromocytoma. Hematologic diseases include, but are not limited to, polycythemia vera. Metabolic diseases include, but are not limited to, gout, Gaucher Disease, Neimann-Pick Disease, Wilson's Disease and hemochromatosis. Infectious diseases include communicable diseases and contagious diseases. Nutritional diseases include, but are not limited to, obesity, anorexia nervosa and bulimia. Toxin- and/or drug- induced diseases include, but are not limited to, hepatitis. Psychiatric diseases include, but are not limited to, addiction to or dependency on substances, schizophrenia, depression and bipolar disorder. Neurologic diseases include, but are not limited to, Alzheimer's disease, Parkinson's disease, multiple sclerosis, myasthenia gravis and amyotrophic lateral sclerosis.

The diseased tissue can be malignant tissue and/or tumor tissue. The tumor can be a metastatic tumor. The tissue can be prostate, urinary bladder, seminoma, germ-cell tumor, ovary, uterine corpus, uterine cervix, testis, kidney, renal pelvis, ureter, urinary organs, vulva, vagina, breast, female genital, penis, male genital, colon, rectum, esophagus, stomach, liver, biliary tract, biliary duct, gallbladder, pancreas, small intestine, anus, anal canal, anorectum, digestive organs, lung, bronchus, respiratory organs, skin, melanoma, brain, spinal cord, peripheral nervous system, central nervous system, eye, orbit, Hodkin lymphoma, non-Hodgkin lymphoma, leukemia, multiple myeloma, oral cavity, pharynx, tongue, mouth, head, neck, thyroid, adrenal, endocrine, larynx, bone, joints, malignancy with unknown primary, neuroblastoma, Wilms tumor, rhabdomyosarcoma, retinoblastoma, osteosarcoma and/or Ewing sarcoma cancerous tissue.

The infection can be caused, for example, by a virus, a bacterium, a parasite, a prion, and/or a fungus.

As used herein, the term “treat” a disease means to reduce or eliminate the disease in a subject, to reduce the number of pathogens causing the disease in the subject, to prevent the disease from spreading in the subject, or to reduce the further spread of the disease in the subject. Similarly, to “treat” an infection means to reduce or eliminate the infection in a subject, to reduce the number of pathogens causing the infection in the subject, to prevent the infection from spreading in the subject, or to reduce the further spread of the infection in the subject. To “treat” an inflammation means to reduce or eliminate the inflammation in a subject.

The subject, for example, can be an immunosuppressed subject or a subject in whom an infection or disease is intractable to treatment using conventional methods. Immunosuppression can occur due to inhibition of one or more components of the immune system due to an underlying disease. Immunosuppression can also be intentionally induced by drugs, for example in patients receiving tissue transplants or in patients with autoimmune disease. Examples of immunosuppressed subjects include, but are not limited to, individuals infected with human immunodeficiency virus (HIV), cancer patients, and organ transplant recipients. HIV causes acquired immune deficiency syndrome (AIDS).

The subject can be a mammal. In different embodiments, the mammal is a mouse, a rat, a cat, a dog, a horse, a sheep, a cow, a steer, a bull, livestock, a primate, a monkey, or preferably a human.

The choice of the particular positron to be used will be influenced by the type of disease to be treated and its localization in the body. Two characteristics are important in the choice of a radioisotope—emission range in the tissue and half-life. Positron emitters which can be used in the therapy of disease include, but are not limited to, the following (half-life and maximum emission energy in parenthesis): ^{52m}Mn (21.1 minutes, 2.63 MeV); ⁶²Cu (9.74 minutes, 2.93 MeV); ⁶⁸Ga (68.1 minutes, 1.9 MeV); ¹¹C (20 minutes, 0.96 MeV); ⁸²Rb (1.2 minutes, 3.35 MeV); ¹¹⁰In (1.15 hours, 2.25 MeV); ¹¹⁸Sb (3.5 minutes, 2.7 MeV); ¹²²I (3.63 minutes, 3.12 MeV); ¹⁸F (1.83 hours, 0.250 MeV); ^{34m}Cl (32.2 minutes, 2.47 MeV); ³⁸K (7.64 minutes, 2.6 MeV); ⁵¹Mn (46.2 minutes, 2.21 MeV); ⁵²Mn (5.59 days, 0.575 MeV); ⁵²Fe (8.28 hours, 0.804 MeV); ⁵⁵Co (17.5 hours, 1.5 MeV); ⁶¹Cu (3.41 hours, 1.22 MeV); ⁷²As (1.08 days, 2.49 MeV); ⁷⁵Br (1.62 hours, 1.74 MeV); ⁷⁶Br (16.2 hours, 3.44 MeV); ^{82m}Rb (6.47 hours, 0.8 MeV); ⁸³Sr (1.35 days, 1.23 MeV); ⁸⁶Y (14.7 hours, 1.25 MeV); ⁸⁹Zr (3.27 days, 0.897 MeV); ^{94m}Tc (52.0 minutes, 2.47 MeV); 120I (1.35 hours, 4.6 MeV); and/or 124I (4.18 days, 2.13 MeV). Preferred positron emitters include ¹⁸F, ⁷⁵Br, ⁷⁶Br, and 124I. All the isotopes listed above in this paragraph emit either positrons or both positrons and Auger electrons. Auger electrons would be useful in therapy in cases where the isotope is delivered to the cell nucleus. ⁶⁴Cu (12.7 hours, 0.657 MeV) is a mixed emitter of beta particles, as well as positrons and Auger electrons.

¹⁸F-labeled compounds have been reviewed in Varagnolo et al., 2000. Br-labeled glucose has been described in Pagani et al., 1997.

Combinations of different positrons can be used, with physical half-lives from about a minute to about 6 days. The method of the present invention can further comprise administering to the subject a plurality of different positron emitters. The different positron emitters can be isotopes of a plurality of different elements. Preferably, the plurality of different positron emitters is more effective in treating the disease, infection and/or inflammation than a single positron emitter within the plurality of different positron emitters, where the radiation dose of the single positron emitter is the same as the combined radiation dose of the plurality of positron emitters.

Routes of administration of positron emitters and agents bound to positron emitters can include, but are not limited to, intravenous, intra-arterial, intra-thecal, intra-peritoneal, intra-muscular, intra-dermal, subcutaneous, oral, and/or localized injection at the site of inflammation, infection or disease, such as intra-tumoral and/or peri-tumoral sites. The positron emitter and/or the agent labeled with the positron emitter can be prepared in a pharmaceutically acceptable sterile vehicle that is injected into the subject. The positron emitter and/or the agent labeled with the positron emitter can be prepared in a pharmaceutically acceptable vehicle that is orally administered to the subject. Systemic administration is preferable, for example, when treating a subject with a tumor that has metastasized.

The dose of the radioisotope can vary depending on the localization and severity of the infection, disease, and/or inflammation, the method of administration of the positron emitter-labeled agent (local or systemic) and the decay scheme of the radioisotope. In order to calculate the doses that can significantly decrease or eliminate disease without radiotoxicity to vital organs, a diagnostic scan of the patient with an agent labeled with diagnostic radioisotope or with the low activity therapeutic radioisotope can be performed prior to therapy, as is customary in nuclear medicine. The dosimetry calculations can be performed using the data from the diagnostic scan (Early et al., 1995; Emami et al. 1991; Hays et al. 2002; Saha, 1998).

Clinical data (Piganelli et al., 1999; Sgouros et al., 1999) indicate that fractionated doses of radiolabeled antibodies and peptides are more effective than single doses against tumors and are less radiotoxic to normal organs. Depending on the status of a patient and the effectiveness of the first treatment with positron emitters, the treatment may consist of one dose or several subsequent fractionated doses.

In one embodiment, the dose of the radioisotope is between about 1 milliCi to about 100 Ci for a 70 kg human. Preferably, the dose of the radioisotope is greater than about 15 milliCi and not more than about 10 Ci for a 70 kg human.

In one embodiment, the agent binds to a σ receptor on malignant tissue and/or tumor cancerous tissue. Sigma receptors are over expressed on many colon, lung, brain, breast, and kidney, as well as other, cancers (Bem et al., 1991). The agent can be α-(4-fluorophenyl)-4-(5-fluoro-2-pyrimidinyl)-1-piperazine-butanol, or an analogue thereof, and the positron emitter-labeled agent can be ¹⁸F-α-(4-fluorophenyl)-4-(5-fluoro-2-pyrimidinyl)-1-piperazine-butanol.

As used herein, an “analogue” is a chemical compound with a structure similar to that of another, but differing from it in respect to one or more components. As used herein, analogues of a chemical compound have similar properties with respect to their ability to bind to and/or be taken up by inflamed, infected and/or diseased tissue, and/or to bind to and/or be taken up by cells at a site of inflamed, infected and/or diseased tissue.

In another embodiment, the cancerous tissue can be breast cancerous tissue, and the agent can bind to an estrogen receptor on breast malignant and/or tumor tissue. The agent can be estradiol or an analogue of estradiol. The positron emitter-labeled agent can be ¹⁸F-fluoro-17β-estradiol.

The diseased tissue can be hypoxic malignant and/or tumor tissue. The agent that binds to the hypoxic tumor can be misonidasole or an analogue of misonidasole. The positron emitter-labeled agent can be ¹⁸F-fluoromisonidazole. Other agents that can bind to the hypoxic tissue are pyruvaldehyde bis(N-methylthiosemicarbazone) and analogues of pyruvaldehyde bis(N-methylthiosemicarbazone). The positron emitter-labeled agent can be ⁶⁴Cu-pyruvaldehyde bis(N-methylthiosemicarbazone).

The agent can be a nucleotide that is taken up by dividing or proliferating cells. The positron emitter-labeled agent can be ¹⁸F-3′-deoxy-3′-fluoro-thymidine (FLT), ¹⁸F-fluorouridine, and/or ¹²⁴I-iododeoxyuridine.

The agent can be an amino acid. The positron emitter-labeled agent can be L-[2-¹⁸F]fluorotyrosine, which can be used, for example, with brain tumors.

The agent can be a gene therapy agent. The positron emitter-labeled agent can be [8-¹⁸F]-fluoroganciclovir.

The agent can be glucose or a glucose analogue, which can be labeled, for example, with ⁷⁵Br, ⁷⁶Br, ¹¹C, ¹²²I, ¹²⁴I and/or ¹²⁰I. The positron emitter-labeled agent can, for example, be a [¹⁸F]fluoro-D-glucose, such as ⁸F-2-deoxy-2-fluoro-D-glucose. This treatment can be used in a variety of conditions, for example, when the subject has a tumor or malignancy, for example breast, lung or esophageal cancer, where the tumor or malignancy takes up the positron-labeled glucose or glucose analogue; when the subject has an infection and the positron-labeled glucose or glucose analogue is taken up by macrophages or other cells at the site of the infection; and/or when the subject has an inflammation and the positron-labeled glucose or glucose analogue is taken up by the inflamed tissue. In regard to the treatment of tumors, this method will be particularly effective in non-mucinous tumors that have a high metabolic consumption of glucose (e.g., melanoma, gliomas, and breast, lung and esophageal tumors).

For example, the invention provides a method for treating a tumor in a human subject, which comprises systemically administering to the subject an amount of glucose labeled with ¹⁸F, or of a glucose analogue labeled with ¹⁸F, effective to treat the tumor, wherein the glucose labeled with ¹⁸F or glucose analogue labeled with ¹⁸F is taken up by the tumor. The positron emitter labeled agent can be [¹⁸F]fluoro-D-glucose (¹⁸F-2-deoxy-2-fluoro-D-glucose), which is preferentially taken up by the tumor. The dose of the radioisotope can be between about 1 milliCi to about 100 Ci for a 70 kg human. Preferably, the dose is greater than about 15 milliCi and not more than about 10 Ci for a 70 kg human. The ratio of standardized uptake value for the tumor to standardized uptake value for the liver can be in a range of about 1.9-3.7. The tumor can be a breast tumor, where the radiation dose to the tumor can be about 220-2200 rad when the radiation dose to the red marrow is about 200 rad, and where the radiation dose to the tumor can be about 330-3300 rad when the radiation dose to the red marrow is about 300 rad. The standardized uptake value for the breast tumor can be in a range of about 1-10. The tumor can be a lung tumor, where the radiation dose to the tumor can be about 1100-3300 rad when the radiation dose to the red marrow is about 200 rad, and where the radiation dose to the tumor can be about 1650-4950 rad when the radiation dose to the red marrow is about 300 rad. The standardized uptake value for the lung tumor can be in the range of about 5-15. The tumor can be an esophageal tumor, where the radiation dose to the tumor can be about 880-7260 rad when the radiation dose to the red marrow is about 200 rad, and where the radiation dose to the tumor can be about 1320-10,890 rad when the radiation dose to the red marrow is about 300 rad. The standardized uptake value for the esophageal tumor can be in the range of about 4-33. Accordingly, the radiation dose to the tumor can be about 220-7260 rad when the radiation dose to the red marrow is about 200 rad, and the radiation dose to the tumor can be about 330-10890 rad when the radiation dose to the red marrow is about 300 rad.

The subject can have a cancer, where expression of a Sodium/Iodide Symporter (NIS) is upregulated in cancerous tissue compared to non-cancerous tissue. ¹²⁴I is administered to treat the subject. ¹²⁴I can be administered in a form that is not bound to a delivery agent.

The cancerous tissue can be a tumor of the adrenal gland and/or neural crest, and the agent can be metaiodobenzylguanidine (MIBG) or an analogue of metaiodobenzylguanidine (MIBG). MIBG localizes in the medulla of the adrenal gland. The agent can be labeled with ¹²⁴I.

The therapy described herein can be combined with methods to reduce the uptake by normal tissue of the agent labeled with the positron emitter. Uptake of ¹⁸F-FDG by cardiac and skeletal muscle is reduced by fasting, and uptake by skeletal muscle can be further reduced with benzodiazepine therapy (Barrington and Maisey, 1996). Corticosteroid therapy can be used to decrease brain inflammation and edema (Patchell et al., 1998). The therapy dose can be also be fractionated to prevent toxicity to the brain. The dose to the bladder and kidneys can be reduced with a Foley catheter and diuretic administration (Morgan et al., 1999).

The invention also provides a method of making a composition effective to treat a disease, infection and/or inflammation in a subject which comprises admixing a positron emitter-labeled agent and a carrier, wherein the agent binds to and/or is taken up by inflamed, infected and/or diseased tissue, and/or binds to and/or is taken up by cells at a site of inflamed, infected and/or diseased tissue. The invention provides a composition comprising an amount of a positron-labeled agent effective to treat a disease, infection and/or inflamation in a subject and a carrier, wherein the agent binds to and/or is taken up by inflamed, infected and/or diseased tissue, and/or binds to and/or is taken up by cells at a site of inflamed, infected and/or diseased tissue. As used herein, the term “carrier” encompasses any of the standard pharmaceutical carriers, such as a sterile isotonic saline, phosphate buffered saline solution, water, and emulsions, such as an oil/water or water/oil emulsions.

The invention also provides a kit comprising a positron emitter and/or an agent labeled with a positron emitter in a container, which is suitable for use with the methods of treatment disclosed herein. The invention also provides a kit comprising, in a container, an agent that binds to and/or is taken up by inflamed, infected and/or diseased tissue, and/or binds to and/or is taken up by cells at a site of inflamed, infected and/or diseased tissue, where the agent is in a form that is to be labeled with a positron emitter.

This invention will be better understood from the Experimental Details which follow. However, one skilled in the art will readily appreciate that the specific methods and results discussed are merely illustrative of the invention as described more fully in the claims which follow thereafter.

### Experimental Details

Materials and Methods

Notch cell line. Mouse mammary gland cell line 5505 was derived from mammary tumors arising in transgenic mice which express an activated form of Notch 4 from the mouse mammary tumor virus long terminal repeat (MMTV-LTR) promoter/enhancer (Fitzgerald et al. 2000). This cell line is further referred to herein as Notch cells. The Notch cells were cultured in DMEM supplemented with 10% FCS, 10% NCTC-109, 1% non-essential amino acids, 4.5 mg/L D-glucose and 1% penicillin-streptomycin.

Animals. All procedures involving mice were conducted in accordance with the National Institutes of Health regulations concerning the use and care of experimental animals. The study of mice was approved by the Albert Einstein College of Medicine (AECOM) Animal Use Committee. Transgenic mice expressing the polyoma middle T antigen (PyMT) in the mammary epithelium under the control of mouse mammary tumor virus (MMTV) long terminal repeat were provided by Dr. W. J. Muller (McMaster University, Ontario, Canada) and then bred at AECOM. At the age of 10 weeks these mice have multiple mammary tumors 0.15-0.17 cm in diameter. In mice older than 18 weeks the tumors are more than 1 cm in diameter. MMTV-NeuT mice (Muller et al. 1988), which carry erbB-2 (Neu), one of the better known human breast cancer oncogenes, were purchased from Jackson Laboratories. MMTV-NeuT mice were obtained as retired breeders and some of them developed mammary tumors at the age of 5 months, whereas others remained-tumor free until 6-7 months of age. C3H/B6 female mice lacking the PyMT transgene were used as healthy controls in therapy experiments. Notch tumor-bearing mice were produced by subcutaneously injecting six to eight week old female nude mice with 105 Notch cells on their right flank. One week after implantation the tumors reached 0.5-0.7 cm in diameter and therapy with FDG was initiated.

Imaging of mice with ¹⁸F-FDG. PyMT mice older than 18 weeks, with tumors more than 1 cm in diameter, MMTV-NeuT tumor-bearing mice (0.5-1 cm in diameter) and tumor-free MMTV-NeuT mice were fasted for 4 hours before the injection of radiotracer. Three animals were used in each group. They were anesthetized with a mixture of 125 mg/kg ketamine and 10 mg/kg xylazine. An injection of 0.5-2.0 mCi ¹⁸F-FDG was administered intraperitoneally (i.p.). The mice rested while anesthetized for an uptake period of 1 hour. A scan was performed on a dedicated PET scanner CPET (Philips). Scans (6 minute emission and 45 second transmission) were obtained. The data were acquired with a zoom of 2 and a slice thickness of 2 mm. Images were reconstructed with an iterative reconstruction algorithm and corrected for attenuation. To reduce volume averaging, a small round region of interest 12 pixels in size was drawn in the center of the tumor and in the center of normal liver. The diameter of the region was 7 mm. The tumor to liver ratios (TLRs) were calculated by the formula TLR=counts per pixel in tumor divided by counts per pixel in liver.

Notch tumor-bearing animals were fasted and anesthetized as described above. An i.p. injection of 0.5 mCi ¹⁸F-FDG was administered. Mice rested while anesthetized for an uptake period of one hour. A scan was performed on a PET-CT scanner (Gemini, Philips). A 6-minute emission scan was obtained, and then at CT scan was obtained (15 mA). The data were acquired with a zoom of 2 and a slice thickness of 2 mm. Images were reconstructed using an iterative reconstruction algorithm, and corrected for attenuation utilizing the CT data. In order to reduce volume averaging, a small round region of interest 12 pixels in size (ROI) was drawn in the center of the tumor and in the center of the contra-lateral normal muscle. The diameter of the region was 7 mm. The tumor to normal muscle ratio (TNR) was calculated according to the formula: TNR=average counts per pixel in tumor/average counts per pixel in normal muscle.

Radiotoxicity studies of ¹⁸F-FDG in healthy mice. After a 4-hour fast, 2 groups of BALB/c mice were treated i.p. with the following doses of ¹⁸F-FDG: group 1 (three mice), 2.5 mCi; group 2 (three mice), 5 mCi; group 3 (three mice), left untreated for control. The mice were monitored for changes in behavior and body weight for 1 month, after which they were killed. Their heart, brain, liver, kidneys, bladder, and bone marrow were removed, fixed in buffered formalin, sectioned, stained with hematoxylin and eosin (H&E), and analyzed histologically.

Treatment of PyMT mice with ¹⁸F-FDG. After a 4-hour fast, three groups of mice were treated i.p. with the following doses of ¹⁸F-FDG: group 1 (five 10-week-old mice), 2-4 mCi; group 2 (three 22-week-old mice), 2 mCi; group 3 (two C3H/B6 mice), 3 mCi to assess radiation damage to healthy breast tissue. Three 10-week-old and two 22-week-old PyMT mice served as untreated controls. At 10 days after treatment, mice were killed. In younger mice, the right abdominal mammary glands were removed for whole mount preparation as described (Gouon-Evans et al., 2000), then fixed in buffered formalin and sectioned. In older mice, the tumors were removed and processed as above. Some sections were stained with H&E; others were analyzed for the presence of apoptotic and necrotic cells.

Treatment of Notch tumor-bearing mice with ¹⁸F-FDG. Notch tumor-bearing mice with tumor sizes of 0.5-0.7 cm in diameter were fasted for 4 hours and treated i.p. with 2.5 mCi ¹⁸F-FDG. The mice rested while anesthetized for an uptake period of one hour. The therapy group consisted of 10 tumor-bearing mice while the control group of 10 animals did not receive any treatment. Animals were monitored for their vital signs and survival for 1 month. Their tumors were measured every 3 days with calipers in three perpendicular planes and the product of the three measurements was multiplied by 0.5 and expressed in cm³. The growth rate for tumors in cm³/day was calculated using the formula: (initial tumor size−tumor size at last measurement while alive)/number of days survived. Kaplan-Meier and Log Rank statistics were used to compare the survival of the mice in the therapy and control groups. The tumor growth rates in the therapy and control groups were compared using the Mann-Whitney Test. SPSS software, version 11.5 was used for statistical calculations.

Apoptosis and necrosis detection. The detection of apoptotic and necrotic cells in the tumors was performed with an Apoptag® Plus apoptosis detection kit (Serologicals) in accordance with the manufacturer's instructions. In brief, 3′-OH termini of DNA fragments, which are the hallmark of apoptosis, were labeled with digoxigenin-modified nucleotides by terminal deoxynucleotidyl transferase. This enzyme selectively detects apoptotic over necrotic cells. The labeled DNA was detected with an anti-digoxigenin antibody, and detection was performed with a chromagen: apoptotic cells were stained brown, and areas of necrosis were stained yellow.

Profile of Notch cells and tumors for expression of GLUT transporters by immunoblot and immunofluorescence. For use in GLUT profiling, Notch cells were grown as above to co-fluency. Nude mice bearing Notch zenografted tumors were sacrificed one week post-implantation to match the time of ¹⁸F-FDG therapy. The GLUT content of the cells and tumors was determined by immunoblot analysis. GLUT 1, 2, 4, and 8 isoform-specific antibodies directed to the unique carboxyl terminal epitopes (12-16 amino acids in length) were used for these studies. Cells/tumor tissue was homogenized and protein obtained as described previously (Katz et al., 1995). Protein concentration was determined by BCA assay (Pierce) with bovine serum albumin as a reference standard. 20-30 μg of protein from the homogenate from each condition was separated on an 8% SDS-PAGE gel. The protein was transferred to Hybond ECL nitrocellulose, stained with Ponceau S to ensure equal loading and transfer. After transfer, filters were blocked with 10% non-fat dry milk in TBST (10 mM Tris buffer, pH 7.4, 150 mM NaCl, 0.1% Tween 20) for 1 hour at room temperature and incubated with affinity purified GLUT1 antibody (1:500-1:1,000 dilution) for one hour. The blot was then incubated with secondary antibodies conjugated to horseradish peroxidase and visualized by enhance chemiluminescence reagent (ECL). Membranes were exposed to Kodak XAR films and subjected to laser scanning densitometry (Molecular Dynamics, Inc., Sunnyvale, Calif.) for quantification. Subsequently, these blots were reacted with antibodies for other GLUT (2, 4 and 8) isoforms. To assess intratumoral expression and localization of GLUT 1 and 8 isoforms, the tumors were fixed in formalin, parafinized and cut into 5 μm thick section. The consecutive sections were double stained with affinity purified GLUT 1 and 8 specific antibodies (Reagan et al., 2001) for immunofluorescence, and with H&E for histological evaluation.

¹⁸F-FDG-PET in breast cancer patients with metastatic disease. Approval was obtained from the Albert Einstein College of Medicine Cancer Center Protocol Review Committee and the Montefiore Medical Center Institutional Review Board. Retrospectively, five consecutive patients with breast cancer and metastatic disease shown on ¹⁸F-FDG-PET scanning were evaluated. After more than 4 hours of fasting, the patients were administered 3.5 mCi of ¹⁸F-FDG, and rested during an uptake period of 45 minutes. The patients then underwent a whole-body scan on the CPET scanner. The scanning protocol consisted of 6 minutes of emission and two rotations of a ¹³⁷Cs source for transmission per bed position, and approximately six bed positions. Images were corrected for attenuation and reconstructed with the iterative method. To reduce volume averaging, a small round region of interest 12 pixels in size was drawn in the center of the tumor and in the center of normal liver. The diameter of the region was 14 mm. Standardized uptake values (SUVs) were calculated for all regions of interest by the formula SUV=(activity in tissue [mCi/g]) divided by (injected activity [mCi]/body weight [g]). The tumor to liver ratios (TLRS) were calculated by the formula TLR=SUV of tumor divided by SUV of liver.

The dose-limiting organ for therapy with ¹⁸F-FDG is the bone marrow. The activity of ¹⁸F-FDG that will deliver doses of 200 rad to the red marrow (less than 5% damage in 5 years) (Early and Sodee, 1995) and also 300 rad were calculated using the Medical Internal Radionuclide Dosimetry formalism. A dose of 300 rad was considered because it causes acceptable and manageable hematologic toxicity in selected patients (Early and Sodee, 1995). The subsequent doses delivered to ¹⁸F-FDG-avid tumors were calculated.

Equations used for dosimetric calculations. Tumor dosimetry is calculated utilizing MIRDOSE3 software (Stabin) and the equation: D=Ã*S, where D is the dose (cGy), Ã is the total activity in the tumor (μCi·h), and S is the MIRDOSE3 S-value (cGy/μCi·h).

The formula for standardized uptake value (SUV) is:

$$SUV = \frac{Activity in tumor ( μ Ci \text{/} g )}{( Injected activity ( μ Ci ) \text{/} Body weight ( g ) )} .$$

The total activity in the tumor Ã (μCi·h)=A₀(μCi)*1/ln(2)* Tₑ(h), where A₀ is the activity in the tumor at time(0), 1/ln(2) is the inverse of the natural log of 2, and Tₑ is the effective half-life of the activity in the tumor.

Utilizing the equations described, the following equation results:
D(cGy)=SUV*188.23 μCi·h/g*tumor mass (g)*S (cGy/μCi·h).

The tumor dose is somewhat lower for smaller tumors, and higher for larger tumors, but can be approximated by the equation: D (cGy)=100*SUV. Selected patients with adequate bone marrow reserve can tolerate 300 cGy to the red marrow (Wiseman et al., 2001), and the administered dose of HD FDG would be 7.5 Ci. The tumor dose would then be represented by the equation: D (cGy)=150*SUV.

Results

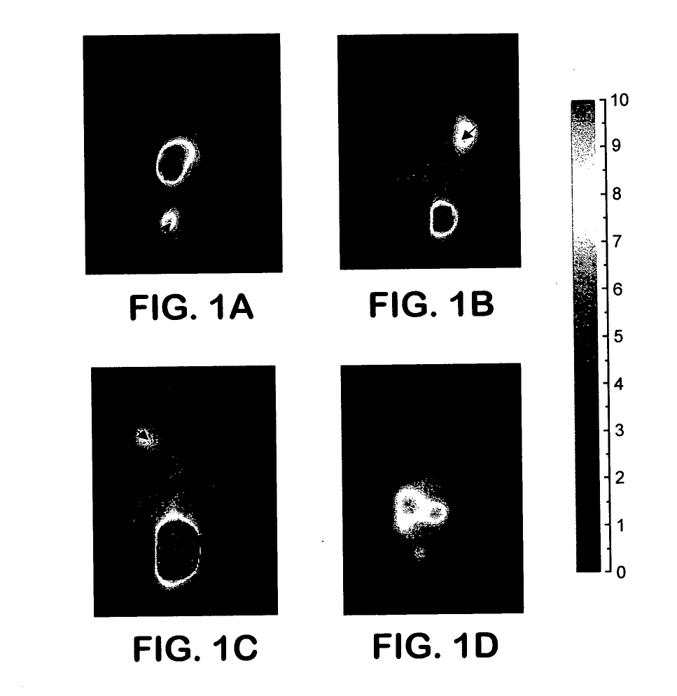

Uptake of ¹⁸F-FDG in mammary tumors ofPyMT and MMTV-neuT mice. Coronal PET images of PyMT and MMTV-neuT mice injected with ¹⁸F-FDG are presented in FIG. 1A-1D. Slices (2 mm thickness) with the highest activity in the tumor were chosen. There was significant uptake of ¹⁸F-FDG by the tumors in PyMT (FIG. 1A, 1B) and MMTV-neuT mice (FIG. 1C). The tumor uptake was uniform. TLRs were (mean±SD) 1.6±0.4 and 1.6±0.1 in MMTV-neuT and PyMT mice, respectively. No abnormal uptake was seen in a tumor-free mouse (FIG. 1D). As ¹⁸F-FDG is normally excreted in the urine, bladder activity was visible in all mice. The avidity of tumors for ¹⁸F-FDG provided an impetus for investigation of the therapeutic effect of ¹⁸F-FDG on mammary tumors in mice.

Lack of radiotoxicity of ¹⁸F-FDG in normal mice. Treatment of healthy mice with doses of up to 5 mCi ¹⁸F-FDG caused no damage in major organs (as determined by histological examination) including bone marrow, which is a dose-limiting organ in patients.

Toleration of treatment of PyMT mice with ¹⁸F-FDG. PyMT mice treated with 2-4 mCi ¹⁸F-FDG were observed for 10 days for obvious signs of radiation toxicity. At 24 hours after injection no radioactivity was detected in mice because of the excretion and complete radioactive decay of the relatively short-lived isotope 18F (half-life 110 minutes). No changes in eating and drinking habits, urine and fecal excretion, or body weight were seen.

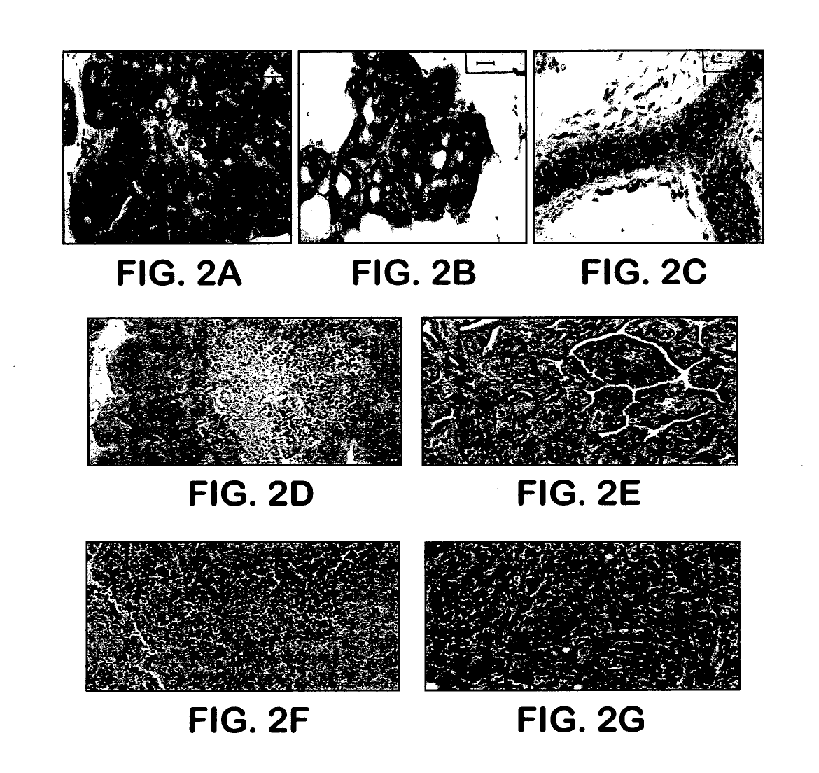

Damage to the tumors by ¹⁸F-FDG. FIG. 2A-2G shows microscopic images of small and large tumors from PyMT mice. The small (0.15-0.17 cm diameter) tumors from 10-week-old mice treated with ¹⁸F-FDG had numerous apoptotic cells clearly present in the center of the treated tumors (FIG. 2A) that were not detected in untreated tumors (FIG. 2B). Overall, 4±1% of cells in the treated small tumors were apoptotic, in comparison with less than 1% in untreated tumors (P<0.05). No apoptosis or necrosis was detected in the healthy mammary glands of C3H/B6 mice treated with 3 mCi of ¹⁸F-FDG (FIG. 2C). ¹⁸F-FDG treatment also caused much more cell death through the necrotic pathway in large (more than 1 cm) tumors in 22-week-old mice than occurred in untreated tumors (FIG. 2D, 2E). In the treated tumors, the areas occupied by necrotic cells were stained brown-yellow (FIG. 2D), as opposed to the sheets of bluish tumor cells in untreated controls (FIG. 2E). Necrotic areas constituted 14% of the tumor volume in 22-week-old mice. Some apoptotic cells were also visible at the rim of the necrotic area.

FIGS. 2F and 2G display H&E-stained large tumors from 22-week-old PyMT mice. Tumors that had been treated with ¹⁸F-FDG were bloody when sectioned, which suggests resorption. This observation was confirmed by heavy infiltration of leukocytes in the H&E-stained section (FIG. 2F). Furthermore, the sections of the tumors treated with ¹⁸F-FDG showed a loss of coherence of the tumor cells with breaks in the epithelial sheets (FIG. 2F) as opposed to solid sheets of tumor cells found in the untreated controls (FIG. 2G).

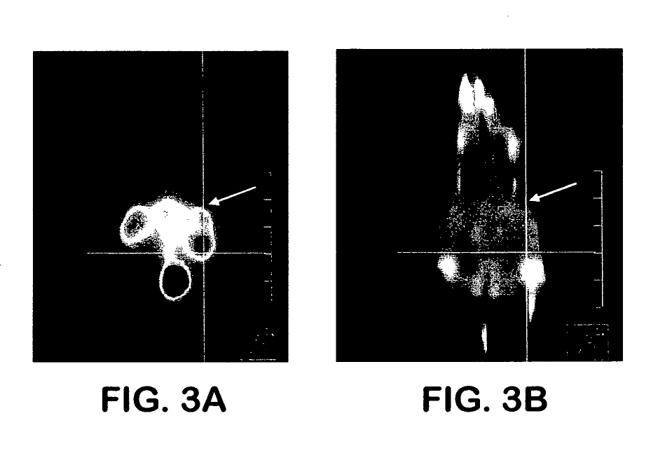

PET-CT imaging of Notch tumor-bearing mice with ¹⁸F-FDG. Coronal PET-CT images of nude mouse xenografted with Notch mammary cancer cells are presented in FIG. 3A-3B. Slices (2 mm thickness) with the highest activity in the tumor were chosen. There was significant uptake of ¹⁸F-FDG by the tumors and the uptake was uniform. The TNR for the Notch tumor was 3.24±0.20.

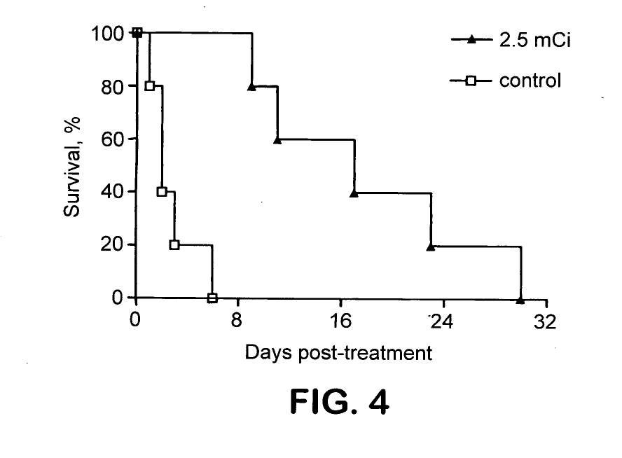

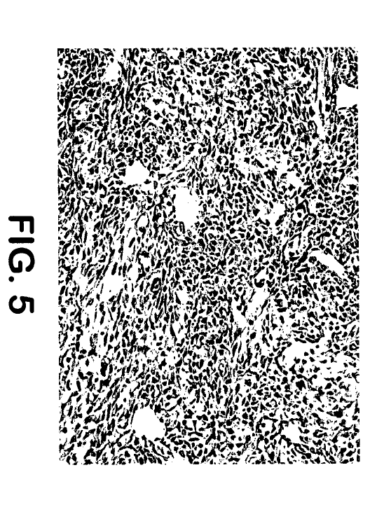

Treatment of Notch tumor-bearing mice with ¹⁸F-FDG. While control mice died within one week post-treatment with a mean (95% Cl) time to death of 3 (2-4) days, mice treated with ¹⁸F-FDG survived significantly longer with a mean time to death of 18 (13-23) days and for periods of up to 30 days (p<0.001, Kaplan-Meier Log Rank). The Kaplan-Meier survival curves of Notch-tumor bearing mice treated with 2.5 mCi ¹⁸F-FDG in comparison with non-treated mice are presented in FIG. 4. The median growth rates of the control and therapy groups and were 0.311 and 0.020 cm³/day respectively (p=0.196, Mann-Whitney). While the median growth rate of the control group was more than one order of magnitude higher than the therapy group, this was not statistically significant as only 2 mice in the control group lived long enough to have their tumor growth monitored. Notch tumors in nude mice were noted to be very aggressive and generate hemangiomas, with subsequent hematoma and death (Fitzgerald et al., 2000). The vascularity of the tumor is seen in the H&E stained section in FIG. 5.

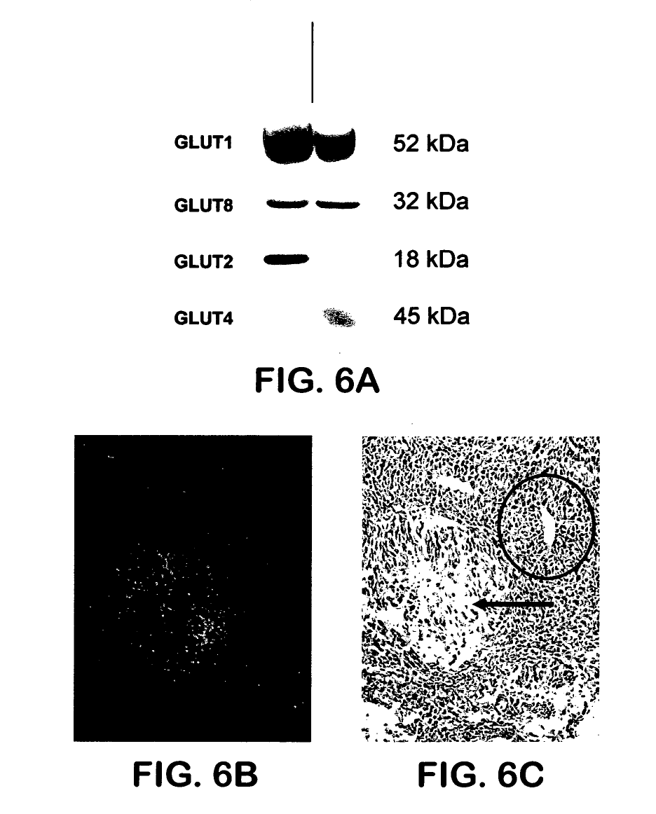

GLUT profile of notch cells and tumors. The Notch-expressing mammary cancer cells and xenografted tumors were analyzed for expression of the GLUT 1, 2, 4 and 8 facilitative glucose transporters using immunoblot technique. FIG. 6A presents the results of GLUT's determination by immunoblot technique in Notch cells (left lane) and in Notch tumor (right lane). High levels of GLUT1 and 8 expression were detected in both cells and tumors.

The immunofluorescence of the Notch tumors double-stained for GLUT 1 and 8 showed that while GLUT 8 was expressed rather homogeneously in the tumors (FIG. 6B), and was seen in viable malignant cells surrounding blood vessels (FIG. 6C, circle), GLUT1 expression was confined to the areas of malignant cells with central necrosis (FIG. 6B) as confirmed by histological analysis of consecutive sections of the tumor (FIG. 6C, arrow).

Human Maximum Tolerated Dose (MTD) of ¹⁸F-FDG. Table 1 indicates the radiation dose (Column 2) to each organ (Column 1) from 1mCi of ¹⁸F-FDG for a 70 Kg person. The tolerated radiation dose of each organ (5% complications in 5 years) is described in Table 1, Column 3. The MTD of ¹⁸F-FDG for each organ is described in Table 1, Column 4. The smallest value in this column is 4.88 Ci, which is the MTD of ¹⁸F-FDG for a 70 Kg person, delivering 200 cGy to the ovaries. This is the critical organ for female patients; however, female patients with metastatic cancer have usually undergone chemotherapy causing ovarian failure or may choose to accept ovarian failure as a side effect of therapy. If ovarian function is not a consideration, then the critical organ is the bone marrow for both male and female patients. A dose of 5.00 Ci of ¹⁸F-FDG would deliver 200 cGy to the bone marrow of a 70 Kg person. If a 70 Kg person were given 5 Ci of ¹⁸F-FDG, the calculated of organ dose is shown in Table 1, Column 5. If a 70 Kg person were given 7.5 Ci of ¹⁸F-FDG, the calculated of organ dose is shown in Table 1, Column 6.

In humans, it is theoretically safe to deliver high dose (HD) ¹⁸F-FDG of 5Ci to a 70 Kg person (200 cGy to marrow), although the dose to the brain of 850 cGy should be fractionated. Selected patients with adequate bone marrow reserve can tolerate 300 cGy to the red marrow (Wiseman et al., 2001), and the MTD dose would increase by 50% to 7.5 Ci.

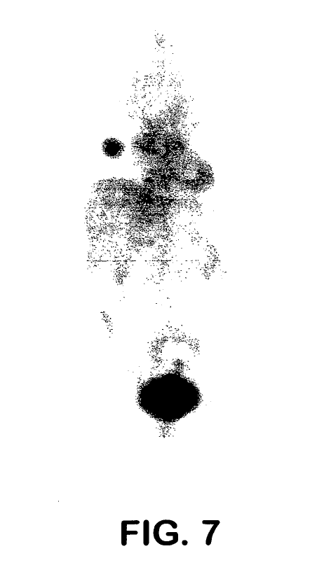

¹⁸F-FDG-PET in breast cancer patients with metastatic disease. Table 2 summarizes the SUVs for metastatic tumors, SUVs for normal liver and TLRs in five patients with metastatic breast cancer imaged with ¹⁸F-FDG. ¹⁸F-FDG-avid metastatic breast cancer in the five retrospectively studied patients showed an average TLR of 2.7±0.8. FIG. 7 presents a ¹⁸F-FDG-PET projection image of a patient with breast cancer, metastatic to the right lung. A left lung metastasis was also seen. Normal activity in the heart, liver, kidneys, bladder, and bowel was observed. It should be also noted that the primary breast cancer had previously been resected, and the residual normal non-lactating breast tissue did not display increased ¹⁸F-FDG uptake over the background. The SUV within a tumor was uniform, because the patients did not display areas of necrosis in the tumors. In patients with multiple tumors the SUVs varied from site to site. In patient 5, the most intense SUV was 9.9 in the tumor with a diameter of 5 cm, and the least intense SUV was 2.9 in the tumor with a diameter of 1.5 cm. In patient 3, the SUV was 5.8 in the tumor with dimensions 10 cm×6 cm×4.5 cm, and the lowest SUV was 3.8 in the tumor with a diameter of 2 cm.

Human Tumor Dosimetry. Table 3 lists data from patients with different types of cancer. For each patient, the tumor with the highest SUVₘₐₓ was used, and corresponding calculated tumor dose (TD) for metastatic cancers are shown in Columns 4 and 5 in Table 3. Tumor dose is calculated by the formulae: D(cGy)=100*SUV, for a dose of 5 Ci HD ¹⁸F-FDG; and D(cGy)=150*SUV, for a dose of 7.5 Ci HD ¹⁸F-FDG.

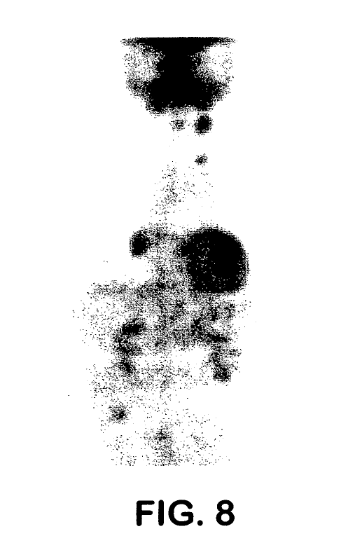

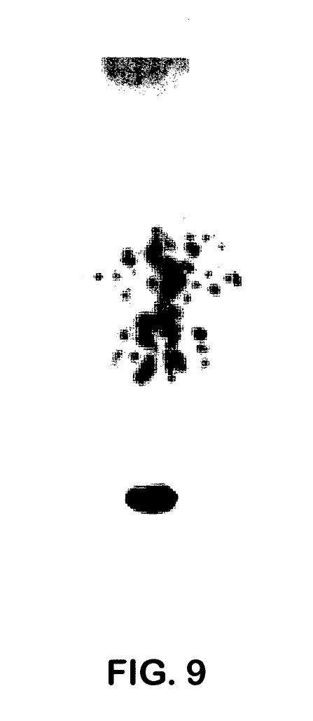

FIG. 8 shows a patient with metastatic non-small cell lung carcinoma (NSCLC). The most intense SUV was 14.8. FDG therapy could deliver 2200 rad to the tumor, if 300 rad were delivered to the bone marrow. FIG. 9 shows a patient with metastatic esophageal carcinoma; the most intense SUV was 33. FDG therapy could deliver 5000 rad to the tumor, if 300 rad were delivered to the bone marrow. These doses are in the tumoricidal range (Toy et al. 2003; Frenken et al. 2001).

The activity of ¹⁸F-FDG to deliver 200 rad to the red marrow in a patient with any type of cancer treated with ¹⁸F-FDG was calculated to be approximately 5.00 Ci for a 70 kg person, and the subsequent tumor doses were calculated to be approximately 100, 500, and 1000 rad for SUVs of 1, 5, and 10, respectively. The activity of ¹⁸F-FDG to deliver 300 rad to the red marrow was calculated to be approximately 7.5 Ci, and tumor doses would be approximately 150, 750, and 1500 rad for SUVs of 1, 5, and 10, respectively. These doses are in the tumoricidal range (Fisher et al. 1985).

While HD ¹⁸F-FDG doses are often not equivalent to external beam radiation therapy (EBRT) doses, HD ¹⁸F-FDG doses are in the same range or order of magnitude as standard EBRT doses (Toy et al., 2003). If treated with the MTD of 7.5 Ci HD ¹⁸F-FDG, 70 Kg patients with metastatic cancers of the breast (SUV=9.9), lung (SUV=14.8) and esophagus (SUV=33) would receive: 1485 cGy, 2220 cGy, and 4950 cGy respectively (FIGS. 7-9).

TABLE 1
Human Maximum Tolerated Dose (MTD) of ¹⁸F-FDG

| | | MTD cGy (5% | | 5 Ci FDG | 7.5 Ci FDG |
|---|---|---|---|---|---|
| | | complications | MTD | Organ dose | Organ dose |
| Organ | cGy/mCi | in 5 years) | FDG (Ci) | (cGy) | (cGy) |
| Brain | 0.17 | 4500 | 26.47 | 850 | 1275 |
| Heart wall | 0.25 | 4000 | 16.00 | 1250 | 1875 |
| Kidneys | 0.078 | 2300 | 29.49 | 390 | 585 |
| Liver | 0.088 | 3000 | 34.09 | 440 | 660 |
| Lungs | 0.056 | 6500 | 116.07 | 280 | 420 |
| Bone marrow | 0.04 | 200 | 5.00 | 200 | 300 |
| Urinary bladder | 0.27 | 6500 | 24.07 | 1350 | 2025 |
| Ovaries | 0.041 | 200 | 4.88 | 205 | 308 |
| Testes | 0.041 | 500 | 12.20 | 205 | 308 |

Column 1: Organs;
Column 2: Radiation dose to organs from 1 mCi ¹⁸F-FDG (cGy/mCi) (Hays et al., 2002);
Column 3: MTD for each organ (cGy) (Emami et al. 1991, Early et al., 1995);
Column 4: Calculated MTD of ¹⁸F-FDG for each organ (Ci);
Column 5: Dose to organs given 5 Ci ¹⁸F-FDG (cGy);
Column 6: Dose to organs given 7.5 Ci ¹⁸F-FDG (cGy).
All values are for a 70 Kg person.

TABLE 2
Standardized uptake values (SUVs) for metastatic tumors and
normal liver; and tumor to liver ratios (TLRs) in patients with
metastatic breast cancer imaged with 3.5 mCi of ¹⁸F-FDG

| | | SUV | | |
|---|---|---|---|---|
| Patient | Diagnosis | Metastasis | Normal liver | TLR |
| 1 | Metastasis to sub- | 5.3 | 2.8 | 1.89 |
| | carinal lymph node | | | |
| 2 | Metastasis to the | 7.4 | 2.3 | 3.36 |
| | left axilla | | | |
| 3 | Liver metastasis | 5.8 | 2.6 | 2.23 |
| 4 | Metastasis to the | 5.3 | 2.1 | 2.52 |
| | right axilla | | | |
| 5 | Right lung metastasis | 9.9 | 2.7 | 3.67 |

SUVs were calculated for all regions of interest by the formula SUV = (activity in tissue [mCi/g] divided by (injected activity [mCi]/body weight [g]).
TLRs were calculated by the formula TLR = SUV of tumor divided by SUV of liver.

TABLE 3
Human Tumor Dosimetry

| | Number | | | Tumor Dose (cGy) | | Tumor Dose (cGy) | |
|---|---|---|---|---|---|---|---|
| Metastatic | of | SUVₘₐₓ | | 5 Ci HD FDG | | 7.5 Ci HD FDG | |
| Cancer | Patients | Median (range) | | Median (range) | | Median (range) | |
| Lung | 10 | 5.9 | (4.9, 14.8) | 590 | (490, 1480) | 885 | (735, 2220) |
| Breast | 10 | 7.2 | (5.3, 12.2) | 720 | (530, 1220) | 1080 | (795, 1830) |
| Head & Neck | 9 | 6.6 | (4.8, 10.2) | 660 | (480, 1020) | 990 | (720, 1530) |
| Esophageal | 5 | 11.1 | (3.9, 33) | 1110 | (390, 3300) | 1665 | (585, 4950) |
| Anaplastic | 3 | 17.4 | (12.8, 19.7) | 1740 | (1280, 1970) | 2610 | (1920, 2955) |
| Thyroid | | | | | | | |
| Ovarian | 9 | 5.3 | (4.4, 16.7) | 530 | (440, 1670) | 795 | (660, 2505) |
| Cervical | 4 | 7.0 | (3.8, 7.8) | 700 | (380, 780) | 1050 | (570, 1170) |
| Melanoma | 7 | 9.0 | (2.0, 22.9) | 900 | (200, 2290) | 1350 | (300, 3435) |
| Lymphoma | 9 | 5.6 | (2.0, 14.3) | 560 | (200, 1430) | 840 | (300, 2145) |

For each cancer type, the SUVₘₐₓ median (range) is displayed for this group of patients.
Tumor doses for 5 Ci and 7.5 Ci HD ¹⁸F-FDG were calculated by the formulae: D(cGy) = 100*SUV and D(cGy) = 150*Suv, respectively.
SUV—Standardized Uptake Value.

### PROPHETIC EXAMPLE

1) Treatment of Cancerous Tissue Expressing Sigma Receptor. Sigma receptors are overexpressed on many colon, lung, brain, breast, and kidney, as well as other, cancers (Bem et al., 1991). An agent that binds to a σ receptor on cancerous tissue can be used to target these tissues. For example, the agent can be α-(4-fluorophenyl)-4-(5-fluoro-2-pyrimidinyl)-1-piperazine-butanol, or an analogue thereof, and the positron emitter-labeled agent can be ¹⁸F-α-(4-fluorophenyl)-4-(5-fluoro-2-pyrimidinyl)-1-piperazine-butanol.

2) Treatment of Breast Cancer Using An Estrogen Receptor Binding Agent. Estrogen receptors are expressed on breast cancer tissue (McGuire et al., 1991). An agent that binds to the estrogen receptor can be used to treat breast cancer. The agent can be, for example, estradiol or an analogue of estradiol. The positron emitter-labeled agent can be ¹⁸F-fluoro-17β-estradiol.

3) Treatment of Hypoxic Tumors. Agents that bind to hypoxic cells within tumors can be used to treat such tumors. The agent can be misonidasole or an analogue of misonidasole, such as ¹⁸F-fluoromisonidazole (Liu et al., 1998). Other agents that can be used to target hypoxic tumor cells are pyruvaldehyde bis(N-methylthiosemicarbazone) and analogues thereof (Lewis et al., 2001).

4) Treatment of Proliferating Cells. Agents that are taken up by dividing cells can be used to treat proliferating cells. For example, the agent can be a nucleotide, and the positron emitter-labeled agent can be ¹⁸F-3′-deoxy-3′-fluoro-thymidine (FLT), ¹⁸F-fluorouridine, and/or ¹²⁴I-iododeoxyuridine (Blasberg et al., 2000; Crawford et al., 1982; Sherley et al., 1988).

5) Treatment of Brain Tumors. The positron emitter-labeled amino acid L-[2-¹⁸F]fluorotyrosine has been used for positron emission tomography (PET) imaging of brain tumors (Inoue et al., 1999). L-[2-¹⁸F]fluorotyrosine can also be used to treat brain tumors.

6) Treatment of Tumors of the Adrenal Gland. Metaiodobenzylguanidine (MIBG) localizes in the medulla of the adrenal gland and in neural crest tumors (Ott et al., 1992). Tumors of the adrenal gland can be treated using metaiodobenzylguanidine (MIBG), or an analogue thereof, labeled with ¹²⁴I.

7) Treatment of Tumors Expressing a Sodium/Iodide Symporter (NIS). NIS is expressed in breast tissue during lactation and breast cancer, but not in normal, non-lactating breast tissue (Tazebay et al., 2000). NIS expression has been detected in 19 carcinoma types, including those of the bladder, cervix, oropharynx, colon, lung, pancreas, prostrate, skin, stomach, ovary and endometrium (Wapnir et al., 2003). In addition, gene therapy has been used to induce tissue-specific expression of NIS in prostate cancer cells (Spitzweg et al., 2000). ¹²⁴I can be used by itself, in the absence of being bound to a delivery agent, to treat cancerous tissues that express NIS.

8) Treatment of Infections by Targeting Macrophages. Macrophages accumulate at sites of infection. Marcophages can be targeted, for example, by their uptake of glucose or an analogue thereof, labeled with ⁷⁵Br, ⁷⁶Br, ¹¹C, ¹⁸F, ¹²²I, ¹²⁴I or ¹²⁰I. As described above, a preferred positron emitter-labeled agent is ¹⁸F-2-deoxy-2-fluoro-D-glucose.

9) Treatment of Inflammation. Inflamed tissue takes up glucose, which means it can targeted be with glucose or an analogue thereof, labeled with ⁷⁵Br, ⁷⁶Br, ¹¹C, ¹⁸F, ¹²²I, ¹²⁴I or ¹²⁰I. As described above, a preferred positron emitter-labeled agent is ¹⁸F-2-deoxy-2-fluoro-D-glucose.

Discussion

This application demonstrates the use of positrons for the treatment of disease, infection, and/or inflammation, using treatment of cancer as a working example. Several prophetic examples are also presented. Theoretically, positrons should kill cancer and other diseased cells in the same manner as electrons. A positron is an antiparticle of an electron. Positrons lose their kinetic energy in the tissue in the same manner as electrons, thereby damaging tissue. This is followed by annihilation which results in the emission of two photons with the energy of 511 keV in opposite directions. However, prior to the present study, the cytocidal potential of positrons had been largely unexplored. Historically, owing to the early success of the therapy of thyroid cancer with 131I (Seidlin et al., 1946), which emits electrons, much of the further development of radiopharmaceuticals for cancer therapy has concentrated on electron-emitting radioisotopes. The cytocidal potential of positrons was described by Stoll and colleagues (2001), who used a balloon filled with the positron-emitter ⁶⁸Ga for coronary artery brachytherapy to prevent restenosis.

Metabolic trapping of ¹⁸F-FDG is an attractive mechanism for delivering radioactivity to tumors, because neoplastic cells have an enhanced rate of glucose utilization (MacKeehan, 1982; Warburg, 1930). This mechanism has been used successfully in ¹⁸F-FDG-PET, which is widely used in nuclear medicine for the diagnosis of oncological diseases. ¹⁸F-FDG has the ability to remain in cancer cells with low efflux rates. With a physical half-life of almost 2 hours, ¹⁸F emits energetic positrons with high abundance (96%) and a path length in tissue of about 0.1-0.2 cm. ¹⁸F-FDG is taken up by cells and phosphorylated by hexokinase to ¹⁸F-FDG-6-phosphate. Because ¹⁸F-FDG is not a substrate for glycolysis and does not undergo further metabolism, it remains trapped in the cell (Gallagher et al., 1978). These qualities made ¹⁸F-FDG an attractive candidate for investigation as an agent for the treatment of breast and other cancers.

The imaging experiments in different mouse mammary cancer models (the PyMT model of murine mammary cancer, the MMTV NeuT model, which carries the erbB-2 human breast cancer oncogenes, and the Notch cell xenografted tumor model) showed that it is possible to use these models for the investigation in vivo of the tumoricidal properties of positrons, because tumor uptake was demonstrated. Apoptosis and necrosis were observed in tumors treated with 18F-FDG. Particulate ionizing radiation is known to cause cell death through both the apoptotic (Knox et al., 1992) and necrotic pathways (Comelissen et al., 2002; Gupta et al., 2002). In small tumors (0.15-0.17 cm in diameter) in PyMT mice treated with ¹⁸F-FDG, the prevalent mode of cell death was apoptosis, whereas in large tumors (more than 1 cm in diameter) radiation induced widespread necrosis. A possible explanation for this difference is that as the tumor diameter increased and there was an exponential increase of the absorbed radiation dose to the tumor, the high doses of radiation to the cell surface led to the focal stimulation of tumor necrosis factor-α expression (Gupta et al., 2002) and a high influx of calcium (Cornelissen et al., 2002), which induced necrosis instead of apoptosis. ¹⁸F-FDG at the 2-4 mCi doses that were used for the therapy of tumor-bearing animals contained 0.001-0.002 mg of unlabelled FDG, which should not cause any pharmacological effect because the level of glucose in the blood at fasting is 0.7 mg/ml (70 mg/dl).

Mice bearing Notch cell xenografted tumors were treated with 2.5 mCi ¹⁸F-FDG, which is a dose equivalent to the physiologic human maximum tolerated dose (MTD). The imaging experiments demonstrated that the xenografts actively took up ¹⁸F-FDG with a TNR of 3.24. There was significant prolongation of survival of treated mice in comparison with non-treated controls. These results are important as they demonstrate the therapeutic potential of ¹⁸F-FDG in a very aggressive fast growing tumor when the treatment is also complicated by the formation of hematomas on the tumor as a result of Notch4 activity in vascular endothelial cells (Fitzgerald et al., 2000). The Notch phenomenon was first identified in 1917 by Thomas Hunt Morgan, and the gene was later sequenced in 1985 (Wharton et al.). The gene encodes a trans-membrane receptor with many epidermal growth factor (EGF) repeats located on the extra-cellular surface. The ligand for this receptor is located on neighboring cells. When the ligand binds to this receptor, the cytoplasmic portion cleaves, enters the nucleus and activates transcription. Notch typically influences cell differentiation and cell-fate during development. Notch needs to partner with another oncoprotein, such as RAS, MYC, adenovirus EIA, human papilloma virus or simian virus 40 large T, to cause cancer. The role of Notch has been demonstrated in breast cancer, as normal cell division is disrupted and symmetric division, or malignant transformation occurs (Clarke et al., 2003). Mouse mammary tumor virus (MMTV) insertion in the Notch-4 locus causes mice to develop poorly differentiated mammary carcinomas (Radtke and Raj, 2003). Notch performs in coordination with the RAS oncogene (Fitzgerald et al., 2000) as a downstream effector of RAS and is upregulated in RAS positive breast cancer. Human breast cancer specimens were studied for Notch, and all were positive, while Notch was absent or barely visible in nearby normal breast tissue (Weijzen et al., 2002). Notch-4 is active during human vascular development of both arteries and capillaries. Its ligand, Delta4, shows high expression in the vasculature of human tumors, and is upregulated by hypoxia (Mailhos et al., 2001; Sullivan and Bicknell, 2003). Notch-4 mammary cancer cells were chosen for this study due to the relevance of Notch and RAS in breast cancer and the aggressive nature of this cell line. These aggressive tumors in nude mice were expected to have high metabolic rates and high expression of GLUT receptors which would transport FDG into these cells.

The expression of different GLUT transporters was expressed in Notch cells and derivate tumors. High levels of GLUT1 and 8 expression have been detected in both cells and tumors. The expression of GLUTs has been investigated in breast cancer cells and breast cancer tissue (Aloj et al., 1999; Brown and Wahl, 1993; Younes et al. 1995, 1997; Zamora-Leon et al., 1996). GLUT1 is found in the cytoplasm and cell membrane of primary tumors and lymph node metastases. Normal breast tissue from the same patients expresses very small amounts of GLUT1. GLUT2 is expressed at the same low level in breast tumor tissue as in normal breast tissue. GLUT5 has also been shown to be expressed in breast tumors (Zamora-Leon et al., 1996). Recently several new members of the extended GLUT family have been shown to be expressed in breast tumors. Specifically, GLUT12 expression was increased in human breast cancer relative to normal breast tissue (Rogers et al., 2003). It is known that neu (a receptor), myc (a transcription factor), and ras (a GTPase) oncogenes affect glucose metabolism in different ways (Kunz-Schughart et al., 2000).

A retrospective study of five patients with metastatic breast cancer imaged with ¹⁸F-FDG provided high SUVs in the tumors (mean tumor to liver ratio=2.7±0.8). High SUVs were also observed for metastatic tumors in patients with non-small cell lung cancer and esophageal cancer. In patients with multiple tumors the SUVs varied from site to site. The tumors with greater uptake of the non-metabolite are affected more than the tumors with less uptake during positron therapy. The SUV of small tumors might suffer from volume averaging in measurement, and these tumors might actually receive a higher dose to the tumor than was measured. The dose-limiting organ for ¹⁸F-FDG administration is the red marrow. Other normal tissues show high ¹⁸F-FDG uptake, but they are relatively radioresistant. There are methods to reduce uptake by normal tissue. Uptake by cardiac and skeletal muscle is routinely reduced by fasting, and uptake by skeletal muscle can be further reduced with benzodiazepine therapy (Barrington and Maisey, 1996). Uptake by brain can be reduced with benzodiazepine or phenobarbital therapy (Foster et al. 1987, Theodore et al. 1986). With an administered dose of 7.50 Ci of ¹⁸F-FDG, the dose to the brain is 1275 rad. In external beam brain radiation therapy, 5040 rad is given in 28 fractions of 180 rad each, accompanied by corticosteroid therapy to decrease brain inflammation and edema (Patchell et al., 1998). The ¹⁸F-FDG therapy dose can be also be fractionated and corticosteroids can be administered to prevent toxicity to the brain. The dose to the bladder and kidneys can be reduced with a Foley catheter and diuretic administration (Morgan et al., 1999). These data suggest that ¹⁸F-FDG therapy can be used safely in ¹⁸F-FDG-avid malignancies.

With regard to long-term toxicity from positron therapy, it has been shown that after large cumulative doses of 131I for metastatic thyroid cancer, the risk of secondary leukemia is 0.4 deaths per 10⁴ patient-year-grays (Edmonds and Smith 1986). Patients with metastatic breast cancer treated with high doses of FDG are expected to have a similar risk for latent leukemia. Patients who require whole-brain radiation therapy generally do not live long enough to attain a secondary malignancy, and there are no data on this.

FDG has the advantages that it has a high tumor to background ratio, easily penetrates into the tumor and homogeneously distributes in the tumor mass unlike radiolabeled antibodies which tend to localize on the periphery of the tumor. This allows the positrons to damage the cancer cells, while normal cells are relatively spared.

One other advantage of using ¹⁸F-FDG as a therapeutic agent in cancer is that up to 29% of glucose utilization in the tumor is due to uptake in non-neoplastic cells, mostly macrophages (Kubota et al., 1992). In a mouse model it has been shown that macrophages contribute to tumor progression and metastasis (Lin et al., 2001). Thus, treatment with ¹⁸F-FDG of metastatic cancer with poor prognosis could be particularly suitable because the macrophages are targeted in addition to the tumor cells.

Positron tumor therapy could also be conducted using other positron-emitters that have longer half-lives and emit higher-energy positrons than those emitted by ¹⁸F. For example, ⁷⁶Br-labeled bromo-D-glucose (Pagani et al., 1997) could potentially deliver a higher radiation dose to the tumor because ⁷⁶Br has a longer half-life (16.2 hours) than ¹⁸F and emits positrons with an energy of 3.44 MeV. Other compounds of interest for positron tumor therapy include ¹²⁴I-labeled iododeoxyuridine (half-life 4.2 days, energy 2.13 MeV), which incorporates into the DNA of cancer cells, and ⁶⁴Cu-pyruvaldehyde bis(N-methylthiosemicarbazone) (half-life 12.7 hours, energy 0.657 MeV) for the treatment of hypoxic tumors.

### REFERENCES

Aloj L, Caraco C, Jagoda E, Eckelman W C, and Neumann R D Glut-1 and hexokinase expression: relationship with 2-fluoro-2-deoxy-D-glucose uptake in A431 and T47D cells in culture, Cancer Res. 59:4709-4714 (1999).
Barrington, S F & Maisey, M N: Skeletal muscle uptake of fluorine-18-FDG: effect of oral diazepam J Nucl Med 1996, 37:1127-1129.
Bem W T, Thomas G E, Mamone J Y, Homan S M, Levy B K, Johnson F E, Coscia C J. Overexpression of sigma receptors in nonneural human tumors. Cancer Res. 1991 15;51(24):6558-62.
Blasberg R G, Roelcke U, Weinreich R, Beattie B, von Ammon K, Yonekawa Y, Landolt H, Guenther I, Crompton N E, Vontobel P, Missimer J, Maguire R P, Koziorowski J, Knust E J, Finn R D, Leenders K L. Imaging brain tumor proliferative activity with [124I]iododeoxyuridine. Cancer Res. 2000 Feb. 1;60(3):624-35.
Bombardieri, E & Crippa, F: PET imaging in breast cancer Q J Nucl Med 2001, 45:245-256.
Brown R S, Goodman T M, Zasadny K R, Greenson J K, Wahl R L. Expression of hexokinase II and Glut-1 in untreated human breast cancer. Nucl Med Biol. 2002 May;29(4):443-53.
Brown R S, Wahl R L Overexpression of Glut-1 glucose transporter in human breast cancer. Cancer, 72: 2979-2985 (1993).
Clarke R B, Anderson E, Howell A, Potten C S. Regulation of human breast epithelial stem cells. Cell Prolif. October 2003; 36 Supplp 1: 45-58.
Comelissen, M, Thierens, H, & De Ridder, L: Interphase death in human peripheral blood lymphocytes after moderate and high doses of low and high LET radiation: an electron microscopic approach Anticancer Res 2002, 22:241-245.
Crawford E J, Friedkin M, Wolf A P, Fowler J S, Gallagher B M, Lambrecht R M, MacGregor R R, Shiue C Y, Wodinsky I, Goldin A. 18F-5-Fluorouridine, a new probe for measuring the proliferation of tissue in vivo. Adv Enzyme Regul. 1982;20:3-22.
Di Chiro, G, De La Paz, R L, Brooks, R A, Sokoloff, L, Komblith, P L, Smith, B H, Patronas, N J, Kufta, C V, Kessler, R M, Johnston, G S, Manning, R G, & Wolf, A P: Glucose utilization of cerebral gliomas measured by [¹⁸F]fluorodeoxyglucose and positron emission tomography Neurology 1982, 32:1323-1329.
Early, P J, Bruce D & Sodee, MD: Principles and Practice of Nuclear Medicine St Louis: Mosby 1995.
Edmonds, C J & Smith, T: The long-term hazards of the treatment of thyroid cancer with radioiodine Br J Radiol 1986, 59:45-51.
Emami et al. Tolerance of normal tissue to therapeutic irradiation. IntJRadOncolBiolPhys. Vol 21 p 109-122, 1991.
Fisher B, Wolmark N, Fisher E R, et al: Lumpectomy an axillary dissection for breast cancer: Surgical, pathological and radiation consideration. World J Surg 9:692-8, 1985.
Fitzgerald K, Harrington A and Leder P Ras pathway signals are required for notch-mediated oncogenesis, Oncogene 19: 4191-4198 (2000)
Foster N L, VanDerSpek A F, Aldrich M S, Berent S, Hichwa R H, Sackellares J C, Gilman S, Agranoff B W. The effect of diazepam sedation on cerebral glucose metabolism in Alzheimer's disease as measured using positron emission tomography. J Cereb Blood Flow Metab. August 1987; 7(4): 415-20.
Frenken M. Best palliation in esophageal cancer: surgery, stenting, radiation, or what? Dis Esophagus. 2001;14(2):120-3.
Gallagher, B M, Fowler, J S, Gutterson, N I, McGregor, R R, Wan, C N, & Wolf, A P: Metabolic trapping as a principal of radiopharmaceutical design: some factors responsible for biodistribution of ¹⁸F-2-deoxy-2-fluoro-D-glucose J Nucl Med 1978, 19:1154-1161.
Gouon-Evans, V, Rothenberg, M E, & Pollard, J W: Postnatal mammary gland development requires macrophages and eosinophils Development 2000, 127:2269-2282.
Gupta, V K, Park, J O, Jaskowiak, N T, Mauceri, H J, Seetharam, S, Weichselbaum, R R, & Posner, M C: Combined gene therapy and ionizing radiation is a novel approach to treat human esophageal adenocarcinoma Ann Surg Oncol 2002, 9:500-504.
Hays M T, Watson E E, Thomas S R, Stabin M. MIRD dose estimate report no. 19: radiation absorbed dose estimates from(18)F-FDG. J Nucl Med. February 2002;43(2):210-14.
Hustinx, R, Benard, F, & Alavi, A: Whole-body FDG-PET imaging in the management of patients with cancer Semin Nucl Med 2002, 32:35-46.
Inoue T, Shibasaki T, Oriuchi N, Aoyagi K, Tomiyoshi K, Amano S, Mikuni M, Ida I, Aoki J, Endo K. 18F alpha-methyl tyrosine PET studies in patients with brain tumors. J Nucl Med. March 1999; 40(3) :399-405.
Katz E B, Stenbit A E, Hatton K, DePinho R, and Charron M J Cardiac and adipose tissue abnormalities but not diabetes in mice deficient in GLUT4, Nature. 377:151-155 (1995).
Knox, S J, Goris, M L, & Wessels, B W: Overview of animal studies comparing radioimmunotherapy with dose equivalent external beam radiation Radiother Oncol 1992, 23:111-117.
Kubota, R, Yamada, S, Kubota, K, Ishiwata, K, Tamahashi, N, & Ido, T: Intratumoral distribution of fluorine-18-fluorodeoxyglucose in vivo : high accumulation in macrophages and granulation tissues studies by microautoradiography J Nucl Med 1992, 33:1972-1980.
Kunz-Schughart L A, Doetsch J, Mueller-Klieser W, and Groebe K. Proliferative activity and tumorigenic conversion: impact on cellular metabolism in 3-D culture, Am J Physiol Cell Physiol. 278:C765-780 (2000).
Laszlo J, Humphreys S, Goldin A (1960) Effects of glucose analogues (2-deoxy-D-glucose, 2-deoxy-D-galactose) on experimental tumors, J. Natl Cancer Inst. 24: 267-280.
Lewis J, Laforest R, Buettner T, Song S, Fujibayashi Y, Connett J, Welch M.Copper-64-diacetyl-bis(N4-methylthiosemicarbazone): An agent for radiotherapy. Proc Natl Acad Sci USA. Jan. 30, 2001;98(3):1206-11.
Lin, E Y, Nguyen, A V, Russell, R G, & Pollard, J W: Colony-stimulating factor 1 promotes progression of mammary tumors to malignancy J Exp Med 2001, 193:727-739.
Liu R S, Yen S H, Chang C P, Chu Y K, Chu L S, Yu S M, Chou K L, Wu L C, Liao S Q, Chen K Y, Yeh S H. The Prognostic Value of [F-18] Fluoromisonidazole in Patients with Nasopharyngeal Carcinoma Receiving Radiation and Concurrent Chemotherapy. Clin Positron Imaging. September 1998; 1 (4):245.
MacKeehan W L: Glycolysis, glutaminolysis and cell proliferation. Cell Biol Int Rep 1982, 6:635-650.
Mailhos C, Modlich U, Lewis J, Harris A, Bicknell R, Ish-Horowicz D. Delta4, an endothelial specific notch ligand expressed at sites of physiological and tumor angiogenesis. Differentiation. December 2001; 69(2-3): 135-44.
Meyer M A, Caday C G, Han Y, Vickers B, Nanda A. Potential radiotherapy of human gliomas with [¹⁸F]fluorodeoxyglucose. Society for Neuroscience Abstracts 22: 948 (Abstract 370.3), 1996.
Meyer M A, Caday C G, Toohey R E. Radiotherapy of hypermetabolic gliomas using 18F-FDG: tumor self dose S value calculations for F18. J. Nucl. Med. 39(5) Suppl.: 184P, 1998.
McGuire A H, Dehdashti F, Siegel B A, Lyss A P, Brodack J W, Mathias C J, Mintun M A, Katzenellenbogen J A, Welch M J. Positron tomographic assessment of 16 alpha-[18F] fluoro-17 beta-estradiol uptake in metastatic breast carcinoma. J Nucl Med. August 1991;32(8): 1526-31.
Moran, J K, Lee, H B, & Blaufox, M D: Optimization of urinary FDG excretion during PET imaging J Nucl Med 1999, 40:1352-1357.
Morgan, T H. The theory of the gene. Am. Nat. 51, 513-544 (1917).
Muller, W J, Sinn, E, Pattengale, P K, Wallace, R, & Leder, P: Single-step induction of mammary adenocarcinoma in transgenic mice bearing the activated c-neu oncogene Cell 1988, 54:105-115.
Ott R J, Tait D, Flower M A, Babich J W, Lambrecht R M. Treatment planning for 131I-mIBG radiotherapy of neural crest tumours using 124I-mIBG positron emission tomography. Br J Radiol. September 1992;65(777):787-91.
Paganelli G., Zoboli S., Cremonesi M. et al Receptor-mediated radionuclide therapy with 90-Y-DOTA-D-Phe-Tyr³-Octreotide: Preliminary report in cancer patients. Cancer Biother. Radiopharm. 14: 477-483, 1999.
Pagani, M, Stone-Elander, S, & Larsson, S A: Alternative positron emission tomography with non-conventional positron emitters: effect of their physical properties on image quality and potential clinical applications Eur J Nucl Med 1997, 24:1301-1327.
Patchell, R A, Tibbs, P A, Regine, W F, Dempsey, R J, Mohiuddin, M, Kryscio, R J, Markesbery, W R, Foon, K A, & Young, B: Postoperative radiotherapy in the treatment of single metastasis to the brain: a randomized trial JAMA1998, 280:1485-1489.
Pessin J E, Bell G I Mammalian facilitative glucose transporter family: structure and molecular regulation. Annu Rev Physiol 54: 911-930 (1992).
Radtke F, Raj K. The role of Notch in tumorigenesis: oncogene or tumour suppressor? Nat Rev Cancer. October 2003;3(10):756-67.
Reagan L P, Gorovits N, Hoskin E K, Alves S E, Katz E B, Grillo C A, Piroli G G, McEwen B S, and Charron M J. Localization and regulation of GLUT×1 glucose transporter in the hippocampus of streptozotocin diabetic rats. Proc Natl Acad Sci USA. 98: 2820-2825 (2001).
Rivenzon-Segal D, Rushkin E, Polak-Charcon S, Degani H. Glucose transporters and transport kinetics in retinoic acid-differentiated T47D human breast cancer cells. Am J Physiol Endocrinol Metab. September 2000;279(3):E508-19.
Rogers S, Docherty S E, Slavin J L, Henderson M A, Best J D. Differential expression of GLUT12 in breast cancer and normal breast tissue. Cancer Lett. Apr. 25, 2003;193(2):225-33.
Saha G. Fundamentals of Nuclear Pharmacy, Fourth Edition. Springer-Verlag, New York. 1998. p 189-200.
Seidlin, S M, Marinelli, L D, & Oshry, E: Radioactive iodine therapy JAAM 1946, 132:838-847.
Sgouros, G., Ballangrud, A. M., Jurcic, J. G., McDevitt, M. R., Humm, J. L., Erdi, Y. E., Mehta, B. M., Finn, R. D., Larson, S. M. and Scheinberg, D. A. 1999. Pharmacokinetics and dosimetry of an alpha-particle emitter labeled antibody: 213Bi-HuM195 (anti-CD33) in patients with leukemia. J. Nucl. Med. 40: 1935-1946.
Sherley J L, Kelly T J. Regulation of human thymidine kinase during the cell cycle. J Biol Chem. Jun. 15, 1988;263(17):8350-8.
Spitzweg C, O'Connor M K, Bergert E R, Tindall D J, Young C Y, Morris J C. Treatment of prostate cancer by radioiodine therapy after tissue-specific expression of the sodium iodide symporter. Cancer Res. Nov. 15, 2000;60(22):6526-30.
Stoll, H P, Hutchins, G D, Winkle, W L, Nguyen, A T, Appledom, C R, Janzen, I, Seifert, H, Rube, C, Schieffer, H, & March, K L: Advantages of short-lived positron-emitting radioisotopes for intracoronary radiation therapy with liquid-filled balloons to prevent restenosis J Nucl Med 2001, 42:1375-1383.
Sullivan D C, Bicknell R. New molecular pathways in angiogenesis. Br J Cancer. Jul. 21, 2003; 89(2): 228-31.
Tazebay U H, Wapnir I L, Levy O, Dohan O, Zuckier L S, Zhao Q H, Deng H F, Amenta P S, Fineberg S, Pestell R G, Carrasco N. The mammary gland iodide transporter is expressed during lactation and in breast cancer. Nat Med. August 2000;6(8):871-8.
Theodore W H, DiChiro G, Margolin R, Fishbein D, Porter R J, Brooks R A. Barbiturates reduce human cerebral glucose metabolism.Neurology. January 1986;36(1):60-4.
Toy E, Macbeth F, Coles B, Melville A, Eastwood A. Palliative thoracic radiotherapy for non-small-cell lung cancer: a systematic review. Am J Clin Oncol. April 2003;26(2):112-20.
Varagnolo L, Stokkel M P M, Mazzi U, and Pauwels E K J. 18-F-labeled radiopharmaceuticals for PET in oncology, excluding FDG. Nucl. Med. Biol. 27: 103-113, 2000.
Wapnir I L, van de Rijn M, Nowels K, Amenta P S, Walton K, Montgomery K, Greco R S, Dohan O, Carrasco N. Immunohistochemical profile of the sodium/iodide symporter in thyroid, breast, and other carcinomas using high density tissue microarrays and conventional sections. J Clin Endocrinol Metab. April 2003;88(4):1880-8.
Warburg, O: The Metabolism of Tumors London: Arnold Constable 1930.
Wharton K A, Johansen K M, Xu T, Artavanis-Tsakonas S. Nucleotide sequence from the neurogenic locus notch implies a gene product that shares homology with proteins containing EGF-like repeats. Cell. December 1985; 43(3 Pt 2): 567-81.
Weijzen S, Rizzo P, Braid M, Vaishnav R, Jonkheer S M, Zlobin A, Osborne B A, Gottipati S, Aster J C, Hahn W C, Rudolf M, Siziopikou K, Kast W M, Miele L. Activation of Notch-1 signaling maintains the neoplastic phenotype in human Ras-transformed cells. Nat Med. September 2002; 8(9): 979-86.
Wiseman G A, White C A, Sparks R B, Erwin W D, Podoloff D A, Lamonica D, Bartlett N L, Parker J A, Dunn W L, Spies S M, Belanger R, Witzig T E, Leigh B R. Biodistribution and dosimetry results from a phase III prospectively randomized controlled trial of Zevalin radioimmunotherapy for low-grade, follicular, or transformed B-cell non-Hodgkin's lymphoma. Crit Rev Oncol Hematol. July-August 2001;39(1-2):181-94.
Younes M, Brown R W, Mody D R, Fernandez L, and Laucirica R GLUT1 expression in human breast carcinoma: correlation with known prognostic markers, Anticancer Res. 15:2895-2898 (1995).
Younes M, Lechago L V, Somoano J R, Mosharaf M, and Lechago J. Immunohistochemical detection of Glut3 in human tumors and normal tissues, Anticancer Res. 17:2747-2750 (1997).
Zamora-Leon S P, Golde D W, Concha II, Rivas C I, Delgado-Lopez F, Baselga J, Nualart F, and Vera J C Expression of the fructose transporter GLUT5 in human breast cancer. Proc Natl Acad Sci USA. 93:1847-1852 (1996).

1-64. (canceled)

65. A method for treating a tumor in a subject, which comprises systemically administering to the subject an amount of glucose labeled with ¹⁸F, or of a glucose analogue labeled with ¹⁸F, effective to treat the tumor, wherein the glucose labeled with ¹⁸F or glucose analogue labeled with ¹⁸F is taken up by the tumor.

66. The method of claim 65, wherein [¹⁸F]fluoro-D-glucose is administered to the subject.

67. The method of claim 65, wherein the ratio of standardized uptake value for the tumor to standardized uptake value for the liver is in a range of about 1.9-3.7.

68. The method of claim 65, wherein the tumor is a breast tumor and wherein the radiation dose to the tumor is about 220-2200 rad and the radiation dose to the red marrow is about 200 rad.

69. The method of claim 65, wherein the tumor is a breast tumor and wherein the radiation dose to the tumor is about 330-3300 rad and the radiation dose to the red marrow is about 300 rad.

70. The method of claim 65, wherein the tumor is a breast tumor and wherein the standardized uptake value for the tumor is in a range of about 1-10.

71. The method of claim 65, wherein the tumor is a breast tumor.

72. The method of claim 65, wherein the tumor is a lung tumor and wherein the radiation dose to the tumor is about 1100-3300 rad and the radiation dose to the red marrow is about 200 rad.

73. The method of claim 65, wherein the tumor is a lung tumor and wherein the radiation dose to the tumor is about 1650-4950 rad and the radiation dose to the red marrow is about 300 rad.

74. The method of claim 65, wherein the tumor is a lung tumor and wherein the standardized uptake value for the tumor is in a range of about 5-15.

75. The method of claim 65, wherein the tumor is a lung tumor.

76. The method of claim 65, wherein the tumor is an esophageal tumor and wherein the radiation dose to the tumor is about 880-7260 rad and the radiation dose to the red marrow is about 200 rad.

77. The method of claim 65, wherein the tumor is an esophageal tumor and wherein the radiation dose to the tumor is about 1320-10,890 rad and the radiation dose to the red marrow is about 300 rad.

78. The method of claim 65, wherein the tumor is an esophageal tumor and wherein the standardized uptake value for the tumor is in a range of about 4-33.

79. The method of claim 65, wherein the tumor is an esophageal tumor.

80. The method of claim 65, wherein the radiation dose to the tumor is about 220-7260 rad and the radiation dose to the red marrow is about 200 rad.

81. The method of claim 65, wherein the radiation dose to the tumor is about 330-10890 rad and the radiation dose to the red marrow is about 300 rad.

82. The method of claim 66, wherein the subject is a human subject.

83. The method of claim 65, wherein the dose of the radioisotope is greater than about 15 milliCi and not more than about 10 Ci for a 70 kg human.

84. (canceled)
